(19) United States
(12) Patent Application Publication
(10) Pub. No.: US 2008/0200727 A1
(43) Pub. Date: Aug. 21, 2008

(54) Method for Producing an Amine

(76) Inventors: Jan Eberhardt, Mannheim (DE); Bram Willem Hoffer, Heidelberg (DE); Ekkehard Schwab, Neustadt (DE); Johann-Peter Melder, Bohl-Iggelheim (DE); Udo Rheude, Otterstadt (DE)

Correspondence Address: CONNOLLY BOVE LODGE & HUTZ, LLP, P O BOX 2207, WILMINGTON, DE, 19899, US

(21) Appl. No.: 11/912,981

(22) PCT Filed: Apr. 26, 2006
(86) PCT No.: PCT/EP06/61842

§ 371 Date: Nov. 28, 2007

(30) Foreign Application Priority Data
Apr. 27, 2005 (DE) 10 2005 019 540.7

(51) Int. Cl. C07C 209/00

(52) U.S. Cl. 564/471

(57) ABSTRACT

Processes for preparing amines comprising reacting an aldehyde and/or ketone with hydrogen and a nitrogen compound in the presence of a heterogeneous catalyst, wherein the heterogeneous catalyst comprises a catalyst packing prepared by applying at least one compound selected from the group consisting of catalytically active metals, compounds of catalytically active metals, and mixtures thereof to a support material selected from the group consisting of woven fabrics, knitted fabrics, foils, and combinations thereof.

The present invention relates to a process for preparing an amine by reacting an aldehyde and/or ketone with hydrogen and a nitrogen compound selected from the group of primary and secondary amines in the presence of a heterogeneous catalyst.

The process products find use, inter alia, as intermediates in the preparation of fuel additives (U.S. Pat. No. 3,275,554; DE-A-21 25 039 and DE-A-36 11 230), surfactants, medicaments and crop protection agents, hardeners for epoxy resins, catalysts for polyurethanes, intermediates for preparing quaternary ammonium compounds, plasticizers, corrosion inhibitors, synthetic resins, ion exchangers, textile auxiliaries, dyes, vulcanization accelerants and/or emulsifiers.

For the preparation of an amine by reacting an aldehyde or ketone with hydrogen and a nitrogen compound, high-pressure processes, for example, are known. Here, the hydrogenating amination is effected over a fixed catalyst bed, for which, for example, metal catalysts with Ni, Pd, Pt, promoters on a support are used. See, for example, DE-A1-21 18 283 (BASF AG).

For the preparation of an amine by hydrogenating amination, low-pressure processes are also known. Here, for example, noble metal catalysts in suspension mode are used. See, for example, for the preparation of dimethylcyclohexylamine (DMCHA) over Pd/C: U.S. Pat. No. 4,521,624 (1985) (pressure range 3.4-40 bar) and CN-A-1 092 061 (1994) (pressure range 10-50 bar).

EP-A-611 137 (Sumitomo Chem. Comp.) relates to the reductive amination of cyclic ketones, in which a corresponding imino compound is prepared in a first stage and is subsequently hydrogenated.

EP-A2-312 253 (Kao Corp.) describes the use of specific copper catalysts in the preparation of N-substituted amines from alcohols or aldehydes.

EP-A1-827 944 (BASF AG) relates to the use of thin-layer catalysts in processes for hydrogenating polyunsaturated C₂₋₈ hydrocarbons.

DE-A1-101 56 813 (BASF AG) relates to a process for selectively synthesizing polyalkyleneamines using thin-layer phosphorus catalysts.

US-A1-2003/0049185 (Air Products) describes an improved reactor with a monolith catalyst and its use in oxidations and hydrogenations, especially of nitro compounds.

EP-A1-1 358 935 (Air Products) relates to supported Ni catalysts with Pd as a promoter and a metal selected from Zn, Cd, Cu and Ag as a promoter. Monolithic support materials are mentioned optionally. The catalysts are used in the amination of alcohols and in the hydrogenation of nitro compounds.

WO-A-99/32529 (Shell) teaches a process for hydrogenating macromolecular organic substrates in the presence of a catalyst having a megapore structure.

It was an object of the present invention to overcome one or more disadvantages of the prior art and to discover an improved, economically viable process for preparing an amine. In particular, the process should include a catalyst of high activity and selectivity (comparable with a suspension catalyst) which features ease of removability from the reaction mixture (analogously to a fixed bed catalyst).

Accordingly, a process has been found for preparing an amine by reacting an aidehyde and/or ketone with hydrogen and a nitrogen compound selected from the group of primary and secondary amines in the presence of a heterogeneous catalyst, wherein the catalyst is a catalyst packing which can be produced by applying at least one catalytically active metal and/or at least one compound of this metal to a woven fabric, a knitted fabric or a foil as a support material.

The advantages of the process according to the invention include good mechanical stability of the thin-layer catalyst or monolith catalyst and a low pressure drop over the catalyst bed, even with increasing running time, the easy handling in the installation and deinstallation of the catalyst, and the simple separation of catalyst and product.

The catalysts used in accordance with the invention have the structure described below.

Support Material

The support material used for the catalysts used in accordance with the invention may be a multitude of foils and woven fabrics, and also knitted fabrics, for example loop-drawingly knitted fabrics. It is possible in accordance with the invention to use woven fabrics with different weave types, such as plain weave, body tissue, Dutch weave, five-shaft satin weave or else other specialty weaves. In one embodiment of the invention, useful woven meshes are woven from weavable metal wires such as iron, spring steel, brass, phosphor bronze, pure nickel, Monel, aluminum, silver, nickel silver, nickel, chromium nickel, chromium steel, nonrusting, acid-resistant and high-temperature-resistant chromium nickel steels, and titanium. The same applies to knitted fabrics, for example loop-drawingly knitted fabrics.

It is likewise possible to use woven fabrics or loop-drawingly knitted fabrics made of inorganic materials such as Al₂O₃ and/or SiO₂.

It is also possible in one embodiment of the invention to use synthetic wires and woven fabrics made of plastics. Examples are polyamides, polyesters, polyvinyls, polyolefins such as polyethylene, polypropylene, polytetrafluoroethylene and other plastics which can be processed to give woven fabrics or knitted fabrics.

Preferred support materials are metal foils or metal fabrics, for example stainless steels having the materials numbers 1.4767, 1.4401, 2.4610, 1.4765, 1.4847, 1.4301, etc. The designation of these materials with the materials numbers specified follows the specifications of the materials numbers in the “Stahleisenliste” [List of Steels], published by Verein Deutscher Eisenhüttenleute, 8th Edition, pages 87, 89 and 106, Verlag Stahleisen mbH, Dusseldorf, 1990. The material of materials number 1.4767 is also known under the name Kanthal.

The metal foils and metal fabrics are particularly suitable since they can be roughened by a heat treatment on the surface before the coating with catalytically active compounds or promoters. To this end, the metallic supports are heated in an oxygenous atmosphere such as air at temperatures of from 400 to 1100° C., preferably from 800 to 1000° C., for from 0.5 to 24 hours, preferably from 1 to 10 hours. In one embodiment of the invention, this pretreatment allows the activity of the catalyst to be controlled or increased.

Coating of the Catalyst Supports

The catalyst supports used in accordance with the invention may, according to the invention, be coated with catalytically active compounds and promoters by means of different processes.

In one embodiment of the invention, the substances active as a catalyst and/or promoter are applied by impregnating the support in bulk (for example according to EP-A1-965 384 (BASF AG)), by electrochemical deposition or deposition in the presence of a reducing agent (electroless deposition).

In one embodiment of the invention, the woven catalyst fabric or the catalyst foil may then be reshaped to monoliths for incorporation into the reactor. In a further embodiment of the invention, the reshaping may also be effected before the application of the active substances or promoters.

In one embodiment of the invention, the catalyst supports usable in accordance with the invention, especially the woven fabrics, knitted fabrics and foils, may be coated with “thin layers” of catalytically active compounds and promoters by means of a vacuum deposition technique. “Thin layers” refer to depositions in the thickness range between a few Å (10⁻¹⁰ m) and a maximum of 0.5 μm. The vacuum deposition techniques employed may, according to the invention, be various processes. Examples are thermal evaporation, flash evaporation, cathode atomization (sputtering) and the combination of thermal evaporation and cathode atomization. The thermal evaporation may be effected by direct or indirect electrical heating.

Evaporation by means of electron beam may likewise be used in accordance with the invention. To this end, the substance to be evaporated is surface-heated with an electron beam in a water-cooled crucible so strongly that even high-melting metals and dielectrics are evaporated. Controlled additions of suitable amounts of reactive gases to the residual gas allow, in one embodiment of the invention, chemical reactions to be brought about by vapor deposition techniques in the layer formation. Suitable reaction control thus allows oxides, nitrides or carbides to be obtained on the support.

The process according to the invention allows the supports, especially the woven fabrics, knitted fabrics and foils, to be subjected to discontinuous or continuous vapor deposition in a vacuum deposition unit. For example, the vapor deposition is effected by heating the catalytically active component or compound to be applied, for example a noble metal, under vacuum at from 10⁻² to 10⁻¹⁰ torr, preferably from 10⁻⁴ to 10⁻⁸ torr, by means of an electron beam so strongly that the metal evaporates out of the water-cooled crucible and precipitates on the support. The woven or knitted support fabric is appropriately arranged in such a way that a maximum portion of the vapor stream condenses on the support. An installed winding machine allows the woven fabrics or knitted fabrics to be coated continuously. Preference is given in accordance with the invention to continuous sputtering in an air to air unit.

Parameters and conditions of vacuum deposition techniques can be taken, for example, from the “Handbook of Thin Film Technology”, publisher: Maissel and Glang, McGraw Hill, New York, 1970, “Thin Film Processes” by J. L. Vossen and B. Kern, Academic Press, New York, and also EP-A-198 435. EP-A2-198 435 relates to the production of a catalyst network packet by subjecting woven stainless steel fabric to vapor deposition with platinum or platinum and rhodium.

In the inventive preparation of the catalysts by vacuum deposition techniques, substantially unordered and disrupted polycrystalline particles should be generated on the support, the predominant portion of the atoms being present within the surface. Thus, it differs from the known vapor deposition techniques in the optics and electronics industry, in which a high purity of the support and vapor deposition materials has to be ensured, and a predetermined condensation temperature on the support and also a certain vapor deposition rate have to be established.

In the process according to the invention, one or more catalytically active compounds or promoters may be applied by vapor deposition.

In one embodiment of the invention, the coatings with catalytically active substsance are preferably in the thickness range from 0.2 nm to 100 nm, more preferably from 0.5 nm to 20 nm, in particular from 3 nm to 7 nm.

In one embodiment of the invention, the catalytically active compounds used are the elements of transition group VIII of the Periodic Table of the Elements, preferably nickel, palladium and/or platinum, in particular palladium.

In one embodiment of the invention, promoters may be present and, according to the invention, may be selected, for example, from the elements of main group III, IV, V, VI and also transition group I, II, III, VI, VII of the Periodic Table of the Elements (Chemical Abstracts Service group notation).

The promoter which is used in one embodiment of the invention is preferably selected from copper, silver, gold, zinc, chromium, cadmium, lead, bismuth, tin, antimony, indium, gallium, germanium, tungsten or mixtures thereof, more preferably silver, indium and germanium, copper, gold, zinc, chromium, cadmium, lead, bismuth, tin, antimony.

The layer thickness of the at least one promoter used in one embodiment of the invention is from 0.1 to 20 nm, preferably from 0.1 to 10 nm, in particular from 0.5 to 3 nm.

Before the application of the catalytically active substance and/or the promoter, the support may be modified by vapor depositon of a layer of an oxidizable metal and subsequent oxidation to form an oxide layer. In one embodiment of the invention, the oxidizable metal used is magnesium, aluminum, silicon, titanium, zirconium, tin or germanium, and also mixtures thereof. The thickness of such an oxide layer is, according to the invention, preferably in the range from 0.5 to 200 nm, more preferably from 0.5 to 50 nm.

The coated support material may be heat-treated after the coating, for example a palladium-coated support material at temperatures in the range from 200 to 800° C., preferably from 300 to 700° C., for, for example, from 0.5 to 2 hours.

After the preparation of the catalyst, it can, if desired or required, be reduced with hydrogen at temperatures of from 20 to 250° C., preferably from 100 to 200° C. This reduction may also preferably be carried out in the reactor itself.

In one embodiment of the invention, the catalysts may be built up systematically, for example in a vapor deposition unit with a plurality of different evaporation sources. For example, an oxide layer or, by reactive evaporation, an adhesive layer may be applied first to the support. On this base layer, it is possible to apply catalytically active components and promoters in several alternating layers by vapor deposition. Admission of a reactive gas into the gas stream in the vapor deposition allows promoter layers composed of oxides and other compounds to be obtained. Heat treatment steps may also be included intermediately or subsequently.

The at least one substance active as a catalyst and/or promoter may also be applied by impregnation.

The catalysts prepared in accordance with the invention by vapor deposition, in particular woven catalyst fabrics, knitted catalyst fabrics and catalyst foils, have very good adhesion strength of the catalytically active compounds and promoters. Therefore, they can be reshaped, cut and, for example, processed to monolithic catalyst elements without the catalytic active compounds and promoters becoming detached. It is possible to use the inventive woven catalyst fabrics, knitted catalyst fabrics and catalyst foils to form catalyst packings of any shape for a reactor, for example flow reactor, a reaction column or distillation column. It is possible to prepare catalyst packing elements with different geometries, as are known from distillation and extraction technology. The examples of advantageous inventive catalyst packing geometries which offer the advantage of a low pressure drop in operation are those of the Montz A3 and Sulzer BX, DX and EX design. One example of an inventive catalyst geometry composed of catalyst foils or expanded metal catalyst foils is that of the Montz BSH type.

The amount of catalyst processed per unit volume, especially amount of woven catalyst fabric, amount of knitted catalyst fabric or amount of catalyst foil, can be controlled within a wide range, which gives rise to differing size of the orifices or channel widths in the woven catalyst fabric, knitted catalyst fabric or in the catalyst foil. Appropriate selection of the amount of woven catalyst fabric, knitted catalyst fabric or catalyst foil per unit volume allows the maximum pressure drop in the reactor, for example flow or distillation reactor, to be adjusted, and the catalyst thus to be adjusted to experimental requirements.

The catalyst used in accordance with the invention preferably has a monolithic form, as described, for example, in EP-A2-564 830. Further suitable catalysts are described in EP-A1-218 124 and EP-A1-412 415.

A further advantage of the monolithic catalysts used in accordance with the invention is good ability to be secured in the reactor bed, so that it can be used very efficiently, for example, in hydrogenations in the liquid phase in liquid-phase mode at high superficial velocity. In contrast, in the case of conventional supported catalysts, there is the risk of vortexing in the catalyst bed, which can lead to possible attrition or decomposition of the shaped bodies. In gas phase hydrogenation, the catalyst packing is resistant to impact or vibrations. No attrition occurs.

Hydrogenation

The above-described catalysts are used in accordance with the invention in a process for preparing an amine by reacting an aldehyde and/or ketone with hydrogen and a nitrogen compound selected from the group of primary and secondary amines.

Use of the inventive catalysts allows these aldehydes and ketones to be converted to the corresponding secondary and tertiary amines with high selectivity and high yield.

The carbonyl compound is aminated preferably in the liquid phase, preferably in two reactors connected in series, a conversion of from 60 to 99% being achieved in the first reactor. The residual conversion is achieved in the second reactor, or it serves as a safety reactor.

In the amination in the liquid phase, an adiabatically operated reactor with or without recycling may be adequate.

In a preferred embodiment, the amination is carried out in a backmixed isothermal reactor and an attached adiabatic reactor.

In one embodiment of the invention, the reaction is carried out in the liquid phase or in a mixed liquid/gas phase having at least 50% by weight of the reaction mixture in the liquid phase.

In one embodiment of the invention, the animation may be carried out in trickle mode or in liquid-phase mode.

In liquid-phase mode, the added hydrogenating hydrogen may be present dissolved in the liquid phase.

The reactors used may, for example, be tubular reactors.

In one embodiment of the invention, the entrance temperature of the reactant mixture in the amination is from −10 to 150° C., preferably from 0 to 120° C., in particular from 0 to 90° C.

In order to ensure the formation of a liquid phase, suitable temperature and pressure parameters have to be seleced within the abovementioned ranges, which is dependent upon the substance mixture used in the particular case.

The nitrogen compound is used preferably in from 0.90 to 100 times the molar amount, in particular in from 1.0 to 10 times the molar amount, of the aldehyde and/or ketone used.

The process according to the invention is preferably carried out at an absolute pressure in the range from 1 to 300 bar, preferably from 1 to 100 bar, more preferably from 1 to 50 bar, particularly preferably from 1 to 30 bar.

The process according to the invention of aldehyde and/or ketone amination is preferably carried out at a temperature in the range from 60 to 200° C., preferably from 80 to 170° C., more preferably from 100 to 150° C.

Preference is given to running with an offgas rate of from 5 to 800 standard cubic meters/h, in particular from 20 to 300 standard cubic meters/h.

The catalyst hourly space velocity is preferably in the range from 0.1 to 2.0 kg, preferably from 0.1 to 1.0 kg, more preferably from 0.2 to 0.6 kg, of aldehyde and/or ketone per liter of catalyst (bed volume) and hour.

It is possible to use higher temperatures, higher overall pressures and higher catalyst hourly space velocities. The pressure in the reactor which arises from the sum of the partial pressures of the aminating agent the aldehyde and/or ketone component and the reaction products formed at the specified temperatures is appropriately increased by injecting hydrogen to the desired reaction pressure.

The water of reaction formed in the course of the reaction generally does not have a disruptive effect on the degree of conversion, the reaction rate, the selectivity and the catalyst lifetime and is therefore appropriately not removed therefrom until the workup of the reaction product, for example by distillation.

Once the reaction effluent has appropriately been decompressed, the excess hydrogen and the excess aminating agent present if appropriate are removed therefrom and the resulting crude reaction product is purified, for example by a fractional rectification. Suitable workup processes are described, for example, in EP-A-1 312 600 and EP-A-1 312 599 (both BASF AG).

Unconverted reactants and suitable by-products which occur if appropriate may be recycled back into the synthesis. In batchwise or continues mode, unconverted reactants may, after condensation of the products in the separator in the cycle gas stream, be passed again over the catalyst bed.

It is possible by the process according to the invention to prepare, for example, amines of the formula I

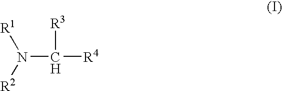

in which

R¹, R² are each hydrogen (H), alkyl such as C₁₋₂₀-alkyl, cycloalkyl such as C₃₋₁₂-cycloalkyl, alkoxyalkyl such as C₂₋₃₀-alkoxyalkyl, dialkylaminoalkyl such as C₃₋₃₀-dialkylaminoalkyl, aryl, aralkyl such as C₇₋₂₀-aralkyl and alkylaryl such as C₇₋₂₀-alkylaryl, or together are —(CH₂)ⱼ—X—(CH₂)ₖ—, (R¹ and R² are not both simultaneously H),

R³, R⁴ are each hydrogen (H), alkyl such as C₁₋₂₀-alkyl, cycloalkyl such as C₃₋₁₂-cycloalkyl, hydroxyalkyl such as C₁₋₂₀-hydroxyalkyl, aminoalkyl such as C₁₋₂₀-aminoalkyl, hydroxyalkylaminoalkyl such as C₂₋₂₀-hydroxyalkylaminoalkyl, alkoxyalkyl such as C₂₋₃₀-alkoxyalkyl, dialkylaminoalkyl such as C₃₋₃₀-dialkylaminoalkyl, alkylaminoalkyl such as C₂₋₃₀-alkylaminoalkyl, R⁵—(OCR⁶R⁷CR⁸R⁹)ₙ—(OCR⁶R⁷), aryl, heteroaryl, aralkyl such as C₇₋₂₀-aralkyl, heteroarylalkyl such as C₄₋₂₀-heteroarylalkyl, alkylaryl such as C₇₋₂₀alkylaryl, alkylheteroaryl such as C₄₋₂₀-alkylheteroaryl, and Y—(CH₂)ₘ—NR⁵—(CH₂)_{q} or, together, —(CH₂)ₗ—X—(CH₂)ₘ— or

R² and R⁴ together are —(CH₂)ₗ—X—(CH₂)ₘ—,

R⁵, R¹⁰ are each hydrogen (H), alkyl such as C₁₋₄-alkyl, alkylphenyl such as C₇₋₄₀-alkylphenyl,

R⁶, R⁷, R⁸, R⁹ are each hydrogen (H), methyl or ethyl,

x is CH₂, CHR⁵, oxygen (O), sulfur (S) or NR⁵,

Y is N(R¹⁰)₂, hydroxyl, C₂₋₂₀-alkylaminoalkyl or C₃₋₂₀-dialkylaminoalkyl,

n is an integer from 1 to 30 and

j, k, l, m, q are each integers from 1 to 4.

The process according to the invention therefore preferably finds use for preparing an amine I by reacting an aidehyde and/or a ketone of the formula VI or VII

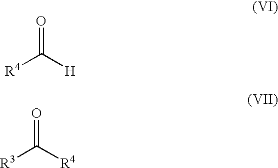

with a nitrogen compound of the formula III

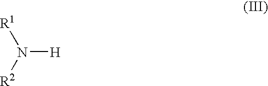

where R¹, R², R³ and R⁴ are each as defined above.

As is evident from the definitions of the R² and R⁴ radicals, the reaction may also be effected intramolecularly in an appropriate amino ketone or amino aldehyde.

To prepare the amine I, in a purely formal sense, a hydrogen atom of the nitrogen compound III is accordingly replaced by the R⁴(R³)CH— radical with release of one molar equivalent of water.

The substituents R¹ to R¹⁰, the variables X, Y, and the indices j, k, l, m, n and q in the compounds I, III, VI and VII are each independently defined as follows:

R¹, R ², R³, R⁴, R⁵, R₆, R⁷, R⁸, R⁹, R¹⁰:
hydrogen (H), (R¹ and R² are not both simultaneously H),

R³, R⁴:
alkyl such as C₁₋₂₀-alkyl, preferably C₁₋₁₄-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, n-hexyl, isohexyl, sec-hexyl, cyclopentylmethyl, n-heptyl, isoheptyl, cyclohexylmethyl, n-octyl, isooctyl, 2-ethylhexyl, n-decyl, 2-n-propyl-n-heptyl, n-tridecyl, 2-n-butyl-n-nonyl and 3-n-butyl-n-nonyl,
hydroxyalkyl such as C₁₋₂₀-hydroxyalkyl, preferably C₁₋₈-hydroxyalkyl, more preferably C₁₋₄-hydroxyalkyl, such as hydroxymethyl, 1-hydroxyethyl, 2-Hydroxyethyl, 1-hydroxy-n-propyl, 2-hydroxy-n-propyl, 3-hydroxy-n-propyl and 1-(hydroxyethyl)ethyl,
aminoalkyl such as C₁₋₂₀-aminoalkyl, preferably C₁₋₈-aminoalkyl, such as aminomethyl, 2-aminoethyl, 2-amino-1,1-dimethylethyl, 2-amino-n-propyl, 3-amino-n-propyl, 4-amino-n-butyl, 5-amino-n-pentyl, N-(2-aminoethyl)-2-aminoethyl and N-(2-aminoethyl)aminomethyl,
hydroxyalkylaminoalkyl such as C₂₋₂₀-hydroxyalkylaminoalkyl, preferably C₃₋₈-hydroxyalkylaminoalkyl, such as (2-hydroxyethylamino)methyl, 2-(2-hydroxyethylamino)ethyl and 3-(2-hydroxyethylamino)propyl,
R⁵—(OCR⁶R⁷CR⁸R⁹)ₙ—(OCR⁶R⁷), preferably R⁵—(OCHR⁷CHR⁹)ₙ—(OCR⁶R⁷), more preferably R⁵—(OCH₂CHR⁹)ₙ—(OCR⁶R⁷),
alkylaminoalkyl such as C₂₋₃₀-alkylaminoalkyl, preferably C₂₋₂₀-alkylaminoalkyl, more preferably C₂₋₈-alkylaminoalkyl, such as methylaminomethyl, 2-methylaminoethyl, ethylaminomethyl, 2-ethylaminoethyl and 2-(isopropylamino)ethyl, (R⁵)HN—(CH₂)_{q},
Y—(CH₂)ₘ—NR⁵—(CH₂)_{q},
heteroarylalkyl such as C₄₋₂₀-heteroarylkyl, such as pyrid-2-ylmethyl, furan-2-ylmethyl, pyrrol-3-ylmethyl and imidazol-2-ylmethyl,
alkylheteroaryl such as C₄₋₂₀-alkylheteroaryl, such as 2-methyl-3-pyridinyl, 4,5-dimethylimidazol-2-yl, 3-methyl-2-furanyl and 5-methyl-2-pyrazinyl,
heteroaryl such as 2-pyridinyl, 3-pyridinyl, 4-pyridinyl, pyrazinyl, pyrrol-3-yl, imidazol-2-yl, 2-furanyl and 3-furanyl,

R¹, R²₁ R³, R⁴:
cycloalkyl such as C₃₋₁₂-cycloalkyl, preferably C₃₋₈-cycloalkyl, such as cyclopropyl, cyclobutyl, cyclopentyl, cyclohexyl, cycloheptyl and cyclooctyl, more preferably cyclopentyl and cyclohexyl,
alkoxyalkyl such as C₂₋₃₀-alkoxyalkyl, preferably C₂₋₂₀-alkoxyalkyl, more preferably C₂₋₈-alkoxyalkyl, such as methoxymethyl, ethoxymethyl, n-propoxymethyl, isopropoxymethyl, n-butoxymethyl, isobutoxymethyl, sec-butoxymethyl, tert-butoxymethyl, 1-methoxyethyl and 2-methoxyethyl, more preferably C₂₋₄-alkoxyalkyl,
dialkylaminoalkyl such as C₃₋₃₀-dialkylaminoalkyl, preferably C₃₋₂₀-dialkylaminoalkyl, more preferably C₃₋₁₀-dialkylaminoalkyl, such as N,N-dimethylaminomethyl, (N,N-dibutylamino)methyl, 2-(N,N-dimethylamino)ethyl, 2-(N,N-diethylamino)ethyl, 2-(N,N-dibutylamino)ethyl, 2-(N,N-di-n-propylamino)ethyl and 2-(N,N-diisopropylamino)ethyl, 3-(N,N-dimethylamino)propyl, (R⁵)₂N—(CH₂)_{q},
aryl such as phenyl, 1-naphthyl, 2-naphthyl, 1-anthryl, 2-anthryl and 9-anthryl, preferably phenyl, 1-naphthyl and 2-naphthyl, more preferably phenyl,
alkylaryl such as C₇₋₂₀-alkylaryl, preferably C₇₋₁₂-alkylphenyl, such as 2-methylphenyl, 3-methylphenyl, 4-methylphenyl, 2,4-dimethylphenyl, 2,5-dimethylphenyl, 2,6-dimethylphenyl, 3,4-dimethylphenyl, 3,5-dimethylphenyl, 2,3,4-trim ethylphenyl, 2,3,5-trimethylphenyl, 2,3,6-trimethylphenyl, 2,4,6-trimethylphenyl, 2-ethylphenyl, 3-ethylphenyl, 4-ethylphenyl, 2-n-propylphenyl, 3-n-propylphenyl and 4-n-propylphenyl,
aralkyl such as C₇₋₂₀-aralkyl, preferably C₇₋₁₂-phenylalkyl, such as benzyl, p-methoxybenzyl, 3,4-dimethoxybenzyl, 1-phenethyl, 2-phenethyl, 1-phenylpropyl, 2-phenylpropyl, 3-phenylpropyl, 1-phenylbutyl, 2-phenylbutyl, 3-phenylbutyl and 4-phenylbutyl, more preferably benzyl, 1-phenethyl and 2-phenethyl,
R³ and R⁴ or R² and R⁴ together are a —(CH₂)ₗ—X—(CH₂)ₘ— group such as —(CH₂)₃—, —(CH₂)₄—, —(CH₂)₅—, —(CH₂)₆—, —(CH₂)₇—, —(CH₂)—O—(CH₂)₂—, —(CH₂)—NR⁵—(CH₂)₂—, —(CH₂)—CHR⁵—(CH₂)₂—, —(CH₂)₂—O—(CH₂)₂—, —(CH₂)₂—NR⁶—(CH₂)₂—, —(CH₂)₂—CHR⁵—(CH₂)₂—, —CH₂—O—(CH₂)₃—, —CH₂—NR⁵—(CH₂)₃—, —CH₂—CHR⁵—(CH₂)₃—,

Rhu 1, R²:
alkyl such as C₁₋₂₀-alkyl, preferably C₁₋₈-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, n-hexyl, isohexyl, sec-hexyl, n-heptyl, isoheptyl, n-octyl, isooctyl, 2-ethylhexyl, more preferably Cl₄-alkyl, or
R¹ and R² together are a —(CH₂)ⱼ—X—(CH₂)₃— group such as —(CH₂)₃—, —(CH₂)₄—, —(CH₂)₅—, —(CH₂)₆—, —(CH₂)₇—, —(CH₂)—O—(CH₂)₂—, —(CH₂)—NR⁵—(CH₂)₂—, —(CH₂)—CHR⁵—(CH₂)₂—, —(CH₂)₂—O—(CH₂)₂—, —(CH₂)₂—NR⁵—(CH₂)₂—, —(CH₂)₂—CHR⁵—(CH₂)₂—, —CH₂—O—(CH₂)₃—, —CH₂—NR⁵—(CH₂)₃—, —CH₂—CHR⁵—(CH₂)₃—,

R⁵, R¹⁰:
alkyl, preferably C₁₋₄-alkyl, such as methyl, ethyl, n-propyl, isopropyl, n-butyl, isobutyl, sec-butyl and tert-butyl, preferably methyl and ethyl, more preferably methyl,
alkylphenyl, preferably C₇₋₄₀-alkylphenyl, such as 2-methylphenyl, 3-methylphenyl, 4-methylphenyl, 2,4-dimethylphenyl, 2,5-dimethylphenyl, 2,6-dimethylphenyl, 3,4-dimethylphenyl, 3,5-dimethylphenyl, 2-, 3-, 4-nonylphenyl, 2-, 3-, 4-decylphenyl, 2,3-, 2,4-, 2,5-, 3,4-, 3,5-dinonylphenyl, 2,3-, 2,4-, 2,5-, 3,4- and 3,5-didecyiphenyl, in particular C₇₋₂₀-alkylphenyl,

R⁶, R⁷, R⁸, R⁹:
methyl or ethyl, preferably methyl,

X:
CH₂, CHR⁵, oxygen (O), sulfur (S) or NR⁵, preferably CH₂ and O,

Y:
N(R¹⁰)₂, preferably NH₂ and N(CH₃)₂,
hydroxyl (OH),
C₂₋₂₀-alkylaminoalkyl, preferably C₂₋₁₆-alkylaminoalkyl, such as methylaminomethyl, 2-methylaminoethyl, ethylaminomethyl, 2-ethylaminoethyl and 2-(isopropylamino)ethyl,
C₃₋₂₀-dialkylaminoalkyl, preferably C₃-₁₆-dialkylaminoalkyl, such as dimethylaminomethyl, 2-dimethylaminoethyl, 2-diethylaminoethyl, 2-(di-n-propylamino)ethyl and 2-(diisopropylamino)ethyl,

j, l:
an integer from 1 to 4 (1, 2, 3 or 4), preferably 2 and 3, more preferably 2,

k, m, q:
an integer from 1 to 4 (1, 2, 3 or 4), preferably 2, 3 and 4, more preferably 2 and 3,

n:
an integer from 1 to 30, preferably an integer from 1 to 8 (1, 2, 3, 4, 5, 6, 7 or 8), more preferably an integer from 1 to 6.

Suitable ketones usable in accordance with the invention are, under the abovementioned prerequisites, virtually all aliphatic and aromatic ketones. The aliphatic ketones may be straight-chain, branched or cyclic; the ketones may comprise heteroatoms. The ketones may further bear substituents or comprise functional groups which behave inertly under the conditions of the hydrogenating amination, for example alkoxy, alkenyloxy, alkylamino or dialkylamino groups, or else, if appropriate, are hydrogenated under the conditions of the hydrogenating amination, for example C-C double or triple bonds. When polyfunctional ketones are to be aminated, it is possible via the control of the reaction conditions to obtain amino ketones, amino alcohols, cyclic amines or polyaminated products.

Preference is given, for example, to aminatingly hydrogenating the following ketones:

Acetone, ethyl methyl ketone, methyl vinyl ketone, isobutyl methyl ketone, butanone, 3-methylbutyl-2-one, diethyl ketone, tetralone, acetophenone, p-methylacetophenone, p-methoxyacetophenone, m-methoxyacetophenone, 1-acetylnaphthalene, 2-acetylnaphthalene, 1-phenyl-3-butanone, cyclobutanone, cyclopentanone, cyclopentenone, cyclohexanone, cyclohexenone, 2,6-dimethylcyclohexanone, cycloheptanone, cyclododecanone, acetylacetone, methyiglyoxal and benzophenone.

Suitable aldehydes usable in accordance with the invention are, under the abovementioned prerequisites, virtually all aliphatic and aromatic aldehydes. The aliphatic aldehydes may be straight-chain, branched or cyclic; the aldehydes may comprise heteroatoms. The aldehydes may further bear substituents or comprise functional groups which behave inertly under the conditions of the hydrogenating amination, for example alkoxy, alkenyloxy, alkylamino or dialkylamino groups, or else, if appropriate, are hydrogenated under the conditions of the hydrogenating amination, for example C-C double or triple bonds. When polyfunctional aldehydes or keto aldehydes are to be aminated, it is possible via the control of the reaction conditions to obtain amino alcohols, cyclic amines or polyaminated products.

Preference is given, for example, to aminatingly hydrogenating the following aldehydes:

formaldehyde, acetaldehyde, propionaldehyde, n-butyraldehyde, isobutyraldehyde, pivalaldehyde, n-pentanal, n-hexanal, 2-ethylhexanal, 2-methylpentanal, 3-methylpentanal, 4-methylpentanal, glyoxal, benzaldehyde, p-methoxybenzaldehyde, p-methylbenzaldehyde, phenylacetaldehyde, (p-methoxyphenyl)acetaldehyde, (3,4-dimethoxyphenyl)acetaldehyde, 4-formyltetrahydropyran, 3-formyltetrahydrofuran, 5-formylvaleronitrile, citronellal, acrolein, methacrolein, ethylacrolein, citral, crotonaldehyde, 3-methoxypropionaldehyde, 3-aminopropionaldehyde, hydroxypivalaldehyde, dimethylolpropionaldehyde, dimethylolbutyraldehyde, furfural, glyoxal, glutaraldehyde and hydroformylated oligomers and polymers, for example hydroformylated polyisobutene (polyisobutenealdehyde) or hydroformylated oligomer obtained by metathesis of 1-pentene and cyclopentene.

The aminating agents used in the hydrogenating amination of aldehydes and/or ketones in the presence of hydrogen may be primary or secondary, aliphatic or cycloaliphatic or aromatic amines.

From di- or oligoaldehydes or di- or oligoketones or keto aldehydes, it is possible by intramolecularly hydrogenating amination to prepare cyclic amines, for example pyrrolidines, piperidines, hexamethyleneimines, piperazines and morpholines.

Preference is given to using the primary or secondary amines as aminating agents to prepare unsymmetrically substituted di- or trialkylamines such as ethyidiisopropylamine and ethyldicyclohexylamine.

Preference is given to using the following mono- and dialkylamines as aminating agents: methylamine, dimethylamine, ethylamine, diethylamine, n-propylamine, di-n-propylamine, isopropylamine, diisopropylamine, isopropylethylamine, n-butylamine, di-n-butylamine, s-butylamine, di-s-butylamine, isobutylamine, n-pentylamine, s-pentylamine, isopentylamine, n-hexylamine, s-hexylamine, isohexylamine, cyclohexylamine, aniline, toluidine, piperidine, morpholine and pyrrolidine.

Amines prepared with particular preference by the process according to the invention are, for example, N,N-di(C₁₄-alkyl)cyclohexylamine (from cyclohexanone and di(C₁₋₄-alkyl)amine), n-propylamines (such as dimethylpropylamine) (from propionaldehyde and DMA), N,N-dimethyl-N-isopropylamine (from acetone and DMA), N,N-dimethyl-N-butylamines (from butanal, i-butanal or butanone and DMA), N-ethyl-N,N-diisopropylamine (from acetaldehyde and N,N-diisopropylamine) and tris(2-ethylhexyl)amine (from 2-ethylhexanal and di(2-ethylhexyl)amine).

All ppm data in this document are based on the weight.

### EXAMPLES

A.

The laboratory experiments were carried out in a 500 ml stirred autoclave from Büchi. The carbonyl compound and the amine to be converted were initially charged in the autoclave in which the particular thin-layer catalyst had been installed. Hydrogen was injected and the mixture was heated to reaction temperature. The stirrer speed was 1200 rpm.

B.

A plant was used which comprised a bubble column equipped with a thin-layer catalyst. The carbonyl compound and the amine to be converted were initially charged in the reactor in which the particular thin-layer catalyst was installed. Hydrogen was injected, and the mixture was heated to reaction temperature. Hydrogen gas and liquid were introduced into the reactor from below. The liquid and gas were pumped in circulation.

The reaction effluents were analyzed by means of gas chromatography (GC) (DB1 separating column, length 60 m; internal diameter 0.32 mm; carrier gas helium; temperature program: 80° C., then at 8° C./minutes to 280° C., finally 15 minutes isothermal at 280° C.).

### Example A1

The experimental autoclave was initially charged with the aldehyde lysmeral and the amine dimethylmorpholine in a molar ratio of 1:1. Isopropanol was added to the reaction mixture (40% by weight based on the total mass of amine and aldehyde). The catalyst used was a thin-layer palladium catalyst based on Kanthal fabric with 490 mg of Pd/m². The noble metal content in the reaction was 0.07% by weight based on Lysmeral. After each reaction, the autoclave and the thin-layer catalyst were flushed with isopropanol.

The fenpropimorph synthesis was carried out at a hydrogen pressure of 6 bar and a reaction temperature of 80° C. After a reaction time of 5 hours, a lysmeral conversion of 76% at a product selectivity of 98% was achieved. In the second and third reaction run, a lysmeral conversion of 78% at a selectivity of 98% was determined under the same reaction conditions after a reaction time of 5 h.

### Example A2

The experimental autoclave was initially charged with lysmeral and dimethylmorpholine in a molar amine:aldehyde ratio of 2.5:1. The reaction was carried out without addition of isopropanol as a solubilizer. The catalyst used was the thin-layer catalyst from example 1. The nobel metal content in the reaction was 0.07% by weight based on lysmeral.

The reaction was carried out at a hydrogen pressure of 14 bar and a reaction temperature of 120° C., After a reaction time of 5 hours, a lysmeral conversion of 93% at a product selectivity of 98% was achieved.

### Example A3

In the same laboratory autoclave as in examples 1 and 2, the same thin-layer catalyst was used to synthesize dimethylcyclohexylamine. Cyclohexanone and dimethylamine were reacted in a molar amine:ketone ratio of 1.2. The noble metal content in the reaction was 0.12% by weight based on the ketone.

The reaction was carried out at a hydrogen pressure of 6 bar and a reaction temperature of 80° C. After a reaction time of 5 hours, a conversion of 33% at a product selectivity of over 98% was determined.

### Example A4

The reaction effluent from example 3 was converted over the thin-layer catalyst from examples 1 to 3 at a hydrogen pressure of 14 bar and a reaction temperature of 120° C. for a 30 further 2.5 h. The cyclohexanone conversion improved to 60% at a product selectivity of 98%.

### Example A5

The experimental autoclave was initially charged with lysmeral and dimethylmorpholine in a 35 molar amine:aldehyde ratio of 2.5:1. The catalyst used was a thin-layer palladium catalyst based on carbon fabric with 1.2 g of Pd/m². The noble metal content in the reaction was 0.14% by weight based on lysmeral.

The reaction was carried out at a hydrogen pressure of 6 bar and a reaction temperature of 80° C. After a reaction time of 6 hours, a lysmeral conversion of 17% at a product selectivity of 98% was achieved. In the second reaction run, a lysmeral conversion of 13% at a selectivity of over 99% was determined under the same reaction conditions after 6 h of reaction.

### Example A6

Lysmeral and dimethylmorpholine were initially charged in the autoclave in a molar amine:galdehyde ratio of 2.5:1. The catalyst used was the thin-layer catalyst from example 5. The noble metal content in the reaction was 0.14% by weight based on lysmeral.

The reaction was carried out at a hydrogen pressure of 14 bar and a reaction temperature of 120° C. After a reaction time of 5 hours, a lysmeral conversion of 49% at a product selectivity of over 99% was achieved.

### Example A7

Cyclohexanone and dimethylamine were reacted in a molar amine:ketone ratio of 1.2. The catalyst used was the thin-film catalyst from example 5. The noble metal content in the reaction was 0.1 6% by weight based on the ketone.

The reaction was carried out at a hydrogen pressure of 6 bar and a reaction temperature of 80° C. After a reaction time of 6 hours, a conversion of 59% at a product selectivity of over 98% was determined.

### Example A8

Cyclohexanone and dimethylamine were reacted in a molar amine:ketone ratio of 1.2. The catalyst used was the thin-film catalyst from example 5. The noble metal content in the reaction was 0.16% by weight based on the ketone.

The reaction was carried out at a hydrogen pressure of 14 bar and a reaction temperature of 120° C. After a reaction time of 6 hours, a conversion of 94% at a product selectivity of over 98% was determined.

### Example B1

The experimental reactor was initially charged with lysmeral and dimethylmorpholine in an amine:aldehyde weight ratio of 1.5:1 (650 ml in total). The catalyst used was a thin-layer palladium catalyst based on Kanthal-900 fabric with 1.96 g of Pd/m². The thin-layer catalyst was preactivated with hydrogen (20 l (STP)/h) (I (STP)=standard liters=volume converted to standard conditions) at 80° C. for one hour. The noble metal content was 0.14% by weight based on lysmeral.

The reaction was carried out at a hydrogen pressure of 6 bar and a liquid inlet temperature of 60° C. The experiment was run with 40-105 liters/h of cycle gas. The cycle liquid flow was 500-1000 g/min. The experiment was run with approx. 50 l (STP)/h of offgas. After a reaction time of 6 hours, a lysmeral conversion of 24% at a product selectivity of over 98% was achieved.

### Example B2

The same experimental reactor as in B1 was initially charged with lysmeral and dimethyl-morpholine in an amine:aldehyde weight ratio of 1.5:1 (650 ml in total). The catalyst used was a thin-film palladium catalyst based on carbon fabric with 0.95 g of Pd/m². The thin-layer catalyst was preactivated with hydrogen (20 l (STP)/h) at 80° C. for 1 hour. The noble metal content in the reaction was 0.07% by weight based on lysmeral.

The reaction was carried out at a hydrogen pressure of 6 bar and a liquid inlet temperature of 60° C. The experiment was run with 40-105 liters/h of cycle gas. The cycle liquid flow was 400-750 g/min. The experiment was run with approx. 50 l (STP)/h of offgas. After a reaction time of 5 hours, a lysmeral conversion of 5% at a product selectivity of over 98% was achieved.

1.-23. (canceled)

24. A process for preparing an amine of formula (I), the process comprising:
providing a reactant selected from the group consisting of aldehydes of formula (VI), ketones of formula (VII), and mixtures thereof; and
reacting the reactant with hydrogen and a nitrogen compound of formula (III) in the presence of a heterogeneous catalyst, wherein the heterogeneous catalyst comprises a catalyst packing prepared by applying at least one compound selected from the group consisting of catalytically active metals, compounds of catalytically active metals, and mixtures thereof to a support material selected from the group consisting of woven fabrics, knitted fabrics, foils, and combinations thereof; 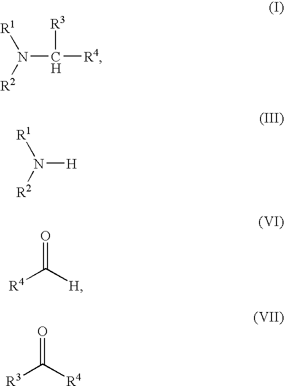
wherein R¹ and R² each independently represent a substituent selected from the group consisting of hydrogen, alkyl groups, cycloalkyl groups, alkoxyalkyl groups, dialkylaminoalkyl groups, aryl groups, aralkyl groups and alkylaryl groups, with the proviso that both R¹ and R² are not simultaneously hydrogen;
wherein R³ and R⁴ each independently represent a substituent selected from the group consisting of hydrogen, alkyl groups, cycloalkyl groups, hydroxyalkyl groups, aminoalkyl groups, hydroxyalkylaminoalkyl groups, alkoxyalkyl groups, dialkylaminoalkyl groups, alkylaminoalkyl groups, R⁵—(OCR⁶R⁷CR⁸R⁹)ₙ—(OCR⁶R⁷), aryl groups, heteroaryl groups, aralkyl groups, heteroarylalkyl groups, alkylaryl groups, alkylheteroaryl groups, and Y—(CH₂)ₘ—NR⁵—(CH₂)_{q}; or R³ and R⁴ together represent —(CH₂)ₗ—X—(CH₂)ₘ—;
wherein R⁵ and R¹⁰ each independently represent a substituent selected from the group consisting of hydrogen, alkyl groups, and alkylphenyl groups;
wherein R⁶, R⁷, R⁸, and R⁹ each independently represent a substituent selected from the group consisting of hydrogen, methyl and ethyl; and
wherein X represents CH₂ or CHR⁵, Y represents N(¹⁰)₂, a hydroxyl, a C₂₋₂₀-alkylaminoalkyl or a C₃₋₂₀-dialkylaminoalkyl, n represents an integer of 1 to 30 and 1, m, and qeach independently represents an integer of 1 to 4.

25. The process according to claim 24, wherein preparing the catalyst packing further comprises applying at least one metal active as a promoter, at least one compound of a metal active as a promoter, or a combination thereof to the support material.

26. The process according to claim 24, wherein applying the compound to the support material comprises vapor deposition or sputtering.

27. The process according to claim 24, wherein applying the compound to the support material comprises impregnation.

28. The process according to claim 24, wherein the catalyst packing comprises at least one monolith which is formed from the support material.

29. The process according to claim 24, wherein the support material comprises a metal, inorganic material or a combination thereof.

30. The process according to claim 24, wherein the support material comprises a metal or inorganic material selected from the group consisting of nickel, aluminum, chromium, titanium, stainless steels, Al₂O₃, SiO₂, plastics and combinations thereof

31. The process according to claim 26, wherein the support material comprises a metal, and prior to applying the compound, the support material is heated to a temperature of 400 to 1100° C. in an oxygenous atmosphere for a period of 0.5 to 24 hours.

32. The process according to claim 25, wherein the catalytically active metals are selected from the elements of the periodic groups 9, 10, and 11; and wherein the metals active as a promoter are selected from the elements of the periodic groups 6, 7, 11, 12, 13, 14, 15, and 16 of the Periodic Table of the Elements (IUPAC notation 1985).

33. The process according to claim 24, wherein the reaction is carried out in the gas phase.

34. The process according to claim 24, wherein the reaction is carried out in the liquid phase or in a mixed liquid/gas phase with at least 50% by weight of the reaction mixture in the liquid phase.

35. The process according to claim 24, wherein the reaciton is carried out at a temperature of 60 to 200° C.

36. The process according to claim 24, wherein the reaction is carried out at an absolute pressure of 1 to 100 bar.

37. The process according to claim 24, wherein the nitrogen compound is present in an amount of 0.90 to 100 times the molar amount of the reactant.

38. The process according to claim 24, wherein the support material comprises a fabric selected from the group consisting of Kanthal and carbon.

39. The process according to claim 24, wherein the catalytically active metal comprises Pd.

40. The process according to claim 25, wherein the catalytically active metal and the metal active as a promoter are not Ni.

41. The process according to claim 25, wherein the catalytically active metal and the metal active as a promoter are not Cu.

42. The process according to claim 24, wherein the nitrogen compound comprises at least one selected from the group consisting of methylamine, dimethylamine, ethylamine, diethylamine, n-propylamine, di-n-propylamine, isopropylamine, diisopropylamine, isopropylethylamine, n-butylamine, di-n-butylamine, s-butylamine, di-s-butylamine, isobutylamine, n-pentylamine, s-pentylamine, isopentylamine, n-hexylamine, s-hexylamine, isohexylamine, and cyclohexylamine.

43. The process according to claim 24, wherein the nitrogen compound comprises at least one selected from N,N-di(C₁₋₄-alkyl)cyclohexylamine, n-propylamines, N,N-dimethyl-N-isopropylamine, N,N-dimethyl-N-butylamines, N-ethyl-N,N-diisopropylamine and tris(2-ethylhexyl)amine.

44. The process according to claim 24, wherein the reactant comprises cyclohexanone and the nitrogen compound comprises dimethylamine.

45. The process according to claim 24, wherein the reactant comprises acetone and the nitrogen compound comprises dimethylamine.

46. The process according to claim 24, wherein the reactant comprises acetaldehyde and the nitrogen compound comprises N,N-diisopropylamine.

47. The process according to claim 24, wherein the reactant comprises 2-ethylhexanal and the nitrogen compound comprises di(2-ethylhexyl)amine.
